(19) United States
(12) Patent Application Publication
(10) Pub. No.: US 2016/0331221 A1
(43) Pub. Date: Nov. 17, 2016

(54) CLEANING DEVICE FOR ENDOSCOPE DISTAL END

(71) Applicant: Michael J. Patzek, IV, Quakertown, PA (US)

(72) Inventor: Michael J. Patzek, IV, Quakertown, PA (US)

(21) Appl. No.: 15/156,198

(22) Filed: May 16, 2016

Related U.S. Application Data
(60) Provisional application No. 62/162,724, filed on May 16, 2015.

(51) Int. Cl. A61B 1/12; B08B 9/032

(52) U.S. Cl. CPC A61B 1/125; B08B 9/0321

(57) ABSTRACT

A device for cleaning an endoscope is disclosed with a hollow body having a distal end, a proximal end and a longitudinal axis therebetween and having a plurality of medially positioned holes through said hollow body. The device may also have an angled partial end positioned adjacent to the distal end. The angled partial end may have an aperture and a connector configured to connect a supply of fluid to flow through said aperture. The aperture may be obliquely angled to direct said supply of fluid into said hollow body. The hollow body may have an opening at the proximal end to allow said endoscope to enter said hollow body and to be cleaned by the flow of fluid through the aperture.

### RELATED APPLICATIONS

This application is related to provisional patent application US 62/162,724 filed on May 16, 2015, and claims international date priority thereof and its subject matter is hereby incorporated herein by reference in its entirety.

### BACKGROUND

1. Technical Field

This disclosure relates to medical cleaning systems, and specifically to cleaning systems for endoscopes or reusable medical devices.

2. Background Art

Endoscopes are valuable tools in the medical industry and are re-used after a cleaning (also known as reprocessing). Traditional reprocessing has multiple steps including manual cleaning in a pre-processing sink, sink or basin using brushes, syringes, and flushing the endoscope with detergents and water as the moving parts are articulated. A pre-process sink has multiple fluid outlets for directing a flow of fluid through the endoscope, or multiple endoscopes at the same time. Cleaning of endoscopes may also be performed in a standard sink or basin utilizing a fluid flushing, pumping device or syringe. Additional steps then include placing the endoscope in a machine for processing, such as an Automated Endoscope Reprocessor (AER).

When an endoscope has multiple moving parts, these can be difficult to access with a cleaning brush, and it can be difficult to properly clean the area around and under the moving parts, such as an elevator/lifter mechanism. One problem with the common cleaning process is that properly cleaning endoscopes is a labor intensive process and requires the endoscopes to go through the entire, time-consuming process in order to be fully cleaned and disinfected so the endoscope can be used for another procedure. Currently endoscopes range in price from $9000.00 to $90,000.00 per endoscope. Endoscopes that have been sent to be processed with ETO for sterilization will not be available for use for 24 hours after the ETO process. Therefore, facilities need to purchase and inventory more endoscopes than they use in a day, enough endoscopes that they can function without each endoscope for 24 hours after each use If facilities had a way to fully clean and disinfect endoscopes in 30 to 50 minutes and return those endoscopes to use, they would not need to purchase and keep so many endoscopes in inventory, resulting in a significant cost savings.

### BRIEF SUMMARY

A device for cleaning an endoscope is disclosed with a hollow body having a distal end, a proximal end and a longitudinal axis there between and having a plurality of medially positioned holes through said hollow body. The device may also have an angled partial end positioned adjacent to the distal end. The angled partial end may have an aperture and a connector configured to connect a supply of fluid to flow through said aperture. The aperture may be obliquely angled to direct said supply of fluid such as detergent solution rinse water and disinfectant into said hollow body. The hollow body may have an opening at the proximal end to allow said endoscope to enter said hollow body and to be cleaned and disinfected by the flow of fluid through the aperture.

### BRIEF DESCRIPTION OF THE DRAWINGS

FIG. 1 is a perspective view of an embodiment of a device for cleaning an endoscope.

FIG. 2 is a side cut-away view of an embodiment of a device for cleaning an endoscope.

FIG. 3 is a perspective view of an embodiment of a device for cleaning an endoscope.

FIG. 4 is a view from the distal end of an embodiment of a device for cleaning an endoscope.

### DETAILED DESCRIPTION

The problems of effective cleaning of endoscopes in a pre-processing sink, a basin or sink and in an automated endoscope reprocessor, including the problem of reaching the critical areas that standard brushing does not reach, can be overcome in whole or in part by use of the disclosed device and method. A device may be used which fits over the end of endoscope and directs a flow of fluid onto the end of the endoscope. A common practice is to wash and disinfect the endoscopes; some facilities will then send the endoscope to be treated with Ethylene Oxide (ETO) to further kill microbes or viruses.

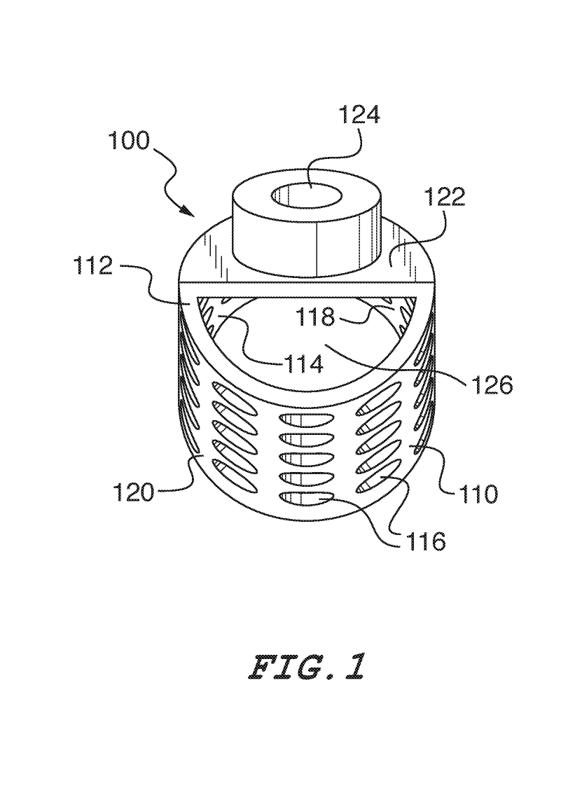

In some embodiments a device 100 for cleaning an endoscope may have a hollow body 110 that fits over the distal end of the endoscope 200 as illustrated in the example embodiment of FIG. 1. The hollow body 110 may have a distal end 112 and a proximal end 114 with medially positioned holes 116 in the hollow body. The hollow body 110 may have an interior 118 and an exterior 120, with the holes 116 allowing fluid to flow from the interior 118 to the exterior 120.

The distal end 112 of the hollow body 110 may have an angled partial end 122 that has an aperture 124. The angled partial end 122 may have a flat portion that is angled in relation to a longitudinal axis of the hollow body 110. The aperture 124 may allow for fluid to flow and be directed at an angle onto the endoscope 200. The angled partial end may allow for a distal end opening 126.

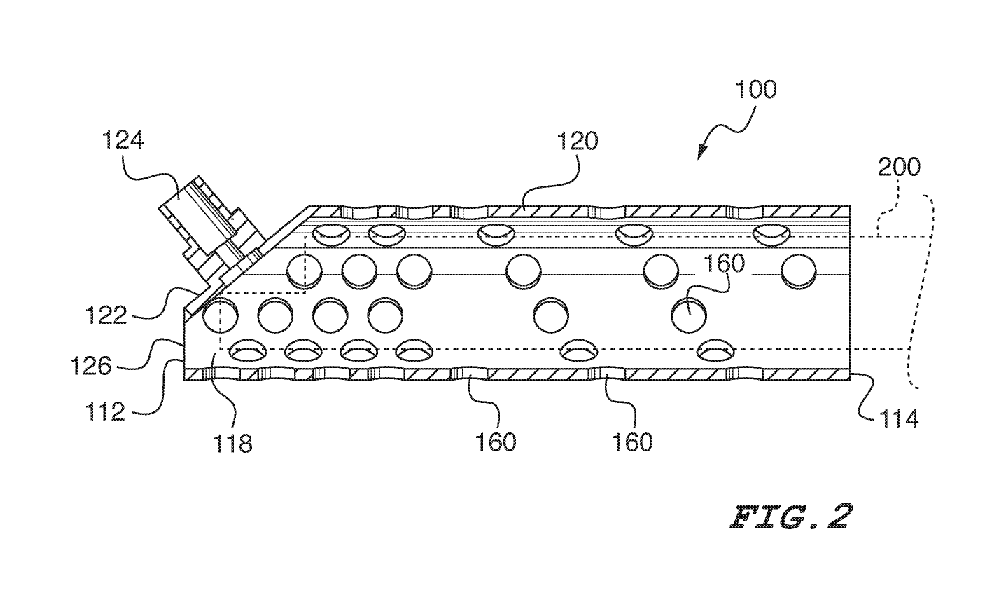

FIG. 2 shows a cut-away side view of an example embodiment of the device 100, and showing the endoscope 200 in dotted lines. In some embodiments the distal end of the endoscope 200 may sit against the angled partial end 122 in the interior 118 of the device 100. There are various makes and models of reusable medical scopes or endoscopes 100 with different configurations. The outline represents one configuration, and other shapes and types of endoscopes or reusable medical scopes may be cleaned with the device 100. In this example, the angled partial end 122 may assist in orienting the endoscope 200 so that fluid flow through the aperture 124 is directed onto the moving parts and instruments in the endoscope 200.

An example method of using the device 100 may include connecting the aperture 124 to a hose or tubing to deliver fluids through the aperture 124, connecting the endoscope 200 to one or more hoses or tubes to deliver fluids through tubing on the interior of the endoscope, and inserting the distal end of the endoscope 200 into the hollow body 110 of the device 100, and orienting the endoscope 200 so that the aperture 124 can direct a flow of fluid onto the instruments on the endoscope 200.

With the endoscope 200 inside the device 100 fluid such as water, detergent, disinfectants, alcohol, air or other fluids used in cleaning, disinfecting and sterilizing, may be directed through the endoscope 200 and through the aperture 124. In a hand cleaning process such as in a pre-processing sink, basin or sink, a user may direct fluid to flow through the endoscope and out the distal end of the endoscope, and also to flow through the aperture 124 and onto the distal end of the endoscope at the same time.

Without the device 100, fluid might only flow through the end of the endoscope, and there may be little or no pressure that will affect the instruments on the end of the endoscope, as the openings on the endoscope allow fluid to pass out of the endoscope, rather than onto the instruments at the distal end of the endoscope. Without the device 100, fluid might not be directed onto the cleaning instruments when the endoscope is flushed or cleaned with cleaning fluids, creating a greater reliance on hand cleaning with brushes or other cleaning methods to ensure proper cleaning of the endoscope.

In some embodiments when using the device 100, the flow from the end of the endoscope may be partially re-directed as the flow interacts with the interior 118 of the hollow body and the interior of the angled partial end 122 so that a greater amount of fluid flows onto and around the distal end of the endoscope 200. Additionally, in some embodiments, the flow of fluid through the aperture 124 also directs cleaning fluid onto the instruments and around the distal end of the endoscope 200.

In some embodiments according to disclosed methods, flow of cleaning fluids may be increased in front of instruments such as an elevator or lifter on the endoscope. The device 100 may also be used in an Automated Endoscope Reprocessor (AER) with the endoscope attached to one or more fluid ports, and the aperture 124 on the device 100 attached to a fluid port to allow cycles of cleaning with detergents, water, disinfectants, alcohol or other solvents, and/or air to flow through the endoscope and around the distal end and instruments on the endoscope 200.

In some embodiments the device 100 is separate from an AER or a pre-processing sink. In other embodiments the device 100 may be connected to or incorporated into the structure of an AER, pre-processing sink, basin, sink, or other machine or cleaning system. In some embodiments the device 100 is detachably connected to a pre-processing sink or AER.

In some embodiments the aperture 124 on the device 100 may be connected to a syringe and the syringe may be used to direct a flow of fluid such as water or detergent onto the distal end of the endoscope 200.

In other embodiments the device 100 may be connected to other machines other than an AER or a pre-processing sink.

In some embodiments the device 100 may be made from stainless steel. In other embodiments the device 100 may be made from other materials such as plastic, aluminum, rubber, or other materials. In some embodiments the hollow body 110 may be made from a mesh or screen material.

The length of the hollow body 110 may vary. In some embodiments the weight of the hollow body may be chosen to be sufficient to hold the endoscope 200 substantially in place relative to the aperture 124 during automated cycles of cleaning. The device 100 may remain in place when fluid pressure acts on the endoscope 200, and the device 100.

The number of holes and placement of holes 116 may vary. The number, position and size of the holes may be selected in some embodiments to allow flow and pressure onto and around the endoscope 200, so that the pressure from the flow does not force the endoscope 200 out of the hollow body 110 of the device 100. Holes 116 in some embodiments may allow fluid to pass through and clean additional parts of the endoscope 200, such as during an automated cleaning cycle where the endoscope 200 and the device 100 may be immersed in a cleaning fluid.

The angle of the angled partial end 122 may vary. In some embodiments the angle of the angled partial end 122 may be selected to direct fluid flow onto the instruments at the distal end of the endoscope 200. In other embodiments the angled end 122 may be selected to direct fluid flow onto other areas or other portions of the endoscope 122. The angled partial end 122 may be at an angle relative to the longitudinal axis of the device 100 of between 15 degrees and 80 degrees. In other embodiments the angle of the angled partial end 122 may be parallel to the longitudinal axis of the device, or perpendicular to the longitudinal axis of the device 100. In some embodiments the angled partial end may be at a 50 degree angle relative to the longitudinal axis of the device 100.

The hollow body 110 is illustrated in the example embodiments as a round cylinder with holes. In other embodiments the hollow body 110 may have other shapes such as a triangular cylinder, a square cylinder, a pentagonal cylinder, or a cylinder having any number of sides.

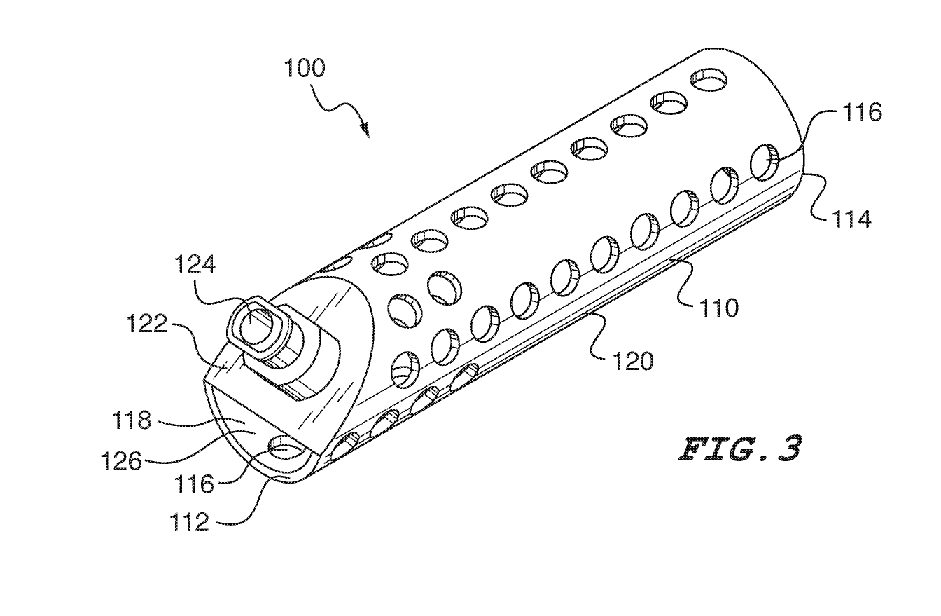

FIG. 3 shows another embodiment of a device 100. The holes 116 may be arranged in various patterns. In some embodiments the holes 116 may be arranged to prevent trapping of air pockets inside the device 100. A trapped air pocket may prevent cleaning fluid from properly cleaning one or more areas of the endoscope. Holes 116 may be arranged to allow debris that comes free from the endoscope 200 (not shown in FIG. 3) to be carried away from the endoscope by the flow of fluids.

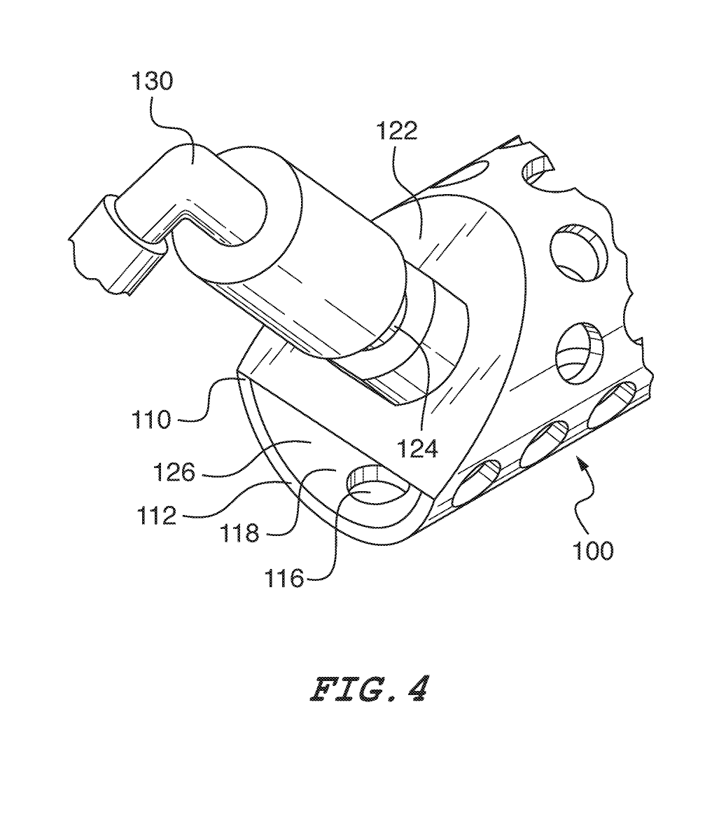

FIG. 4 shows an embodiment of a device 100 that is connected to tubing 130. The tubing 130 may be attached to a pre-processing sink, an AER, a syringe, or other source of fluid to deliver fluid through the tubing 130 and through the aperture 124. In the illustration of FIG. 4 an example endoscope 200 is positioned inside the device 100 and can be seen through the opening 126 at the distal end 112 of the device 100. The aperture 124 may have a connector for connecting a supply of fluid. In some embodiments the connector is a quick connect device. The aperture 124 may have a connector that matches connectors found in hospital settings and used on syringes and medical tubing. In some embodiments the aperture 124 has a female Luer connector.

While the principles of the invention have been made clear in illustrative embodiments, there will be immediately obvious to those skilled in the art many modifications of structure, arrangement, proportions, and methods, the elements, materials, and components used in the practice of the invention, and otherwise, which are particularly adapted to specific environments and operative requirements without departing from those principles. The appended claims are intended to cover and embrace any and all such modifications, within the limits only of the true spirit and scope of the invention.

What is claimed is:

1. A device for cleaning an endoscope comprising:
A hollow body having a distal end, a proximal end and a longitudinal axis therebetween and having a plurality of medially positioned holes through said hollow body;
An angled partial end positioned adjacent to the distal end, said angled partial end having an aperture and a connector configured to connect a supply of fluid to flow through said aperture, wherein said aperture is obliquely angled to direct said supply of fluid into said hollow body;
Wherein said hollow body has an opening at the proximal end to allow said endoscope to enter said hollow body.

2. A method of cleaning an endoscope comprising:
Providing a device comprising:
A hollow body having a distal end, a proximal end and a longitudinal axis therebetween and having a plurality of medially positioned holes through said hollow body;
An angled partial end positioned adjacent to the distal end, said angled partial end having an aperture and a connector configured to connect a supply of fluid to flow through said aperture, wherein said aperture is obliquely angled to direct said supply of fluid into said hollow body;
Wherein said hollow body has an opening at the proximal end to allow said endoscope to enter said hollow body;
Inserting said endoscope into said hollow body;
Connecting said supply of fluid to said aperture; and
Connecting said supply of fluid to said endoscope.
